(19) United States
(12) Patent Application Publication
(10) Pub. No.: US 2007/0299394 A1
(43) Pub. Date: Dec. 27, 2007

(54) Apparatus for Removing Needle Assembly

(76) Inventors: Steven Mark Guy Rolfe, Oxfordshire (GB); Christopher William Hudson, Oxfordshire (GB)

Correspondence Address: JOHN S. PRATT, ESQ;KILPATRICK STOCKTON, LLP, 1100 PEACHTREE STREET, ATLANTA, GA, 30309, US

(21) Appl. No.: 11/587,411

(22) PCT Filed: Apr. 27, 2005
(86) PCT No.: PCT/GB05/50058

§ 371 Date: Jul. 19, 2007

(30) Foreign Application Priority Data
Apr. 27, 2004 (GB) 0409354.8

(51) Int. Cl. A61M 5/50; A61M 5/32

(52) U.S. Cl. 604/110; 604/192

(57) ABSTRACT

Apparatus for removing a needle assembly from an injecting device, the needle assembly comprising a needle holder portion and a needle portion secured to, or integral with, the needle holder portion, the needle assembly being attachable to the injecting device via the needle holder portion, the apparatus comprising a housing (2) having an opening (5, 10, 12) through which the needle assembly can enter the interior of the housing, wherein within the housing there is provided a receiving portion (20) which is arranged to receive the needle holder portion in such a way as to impede rotation of the needle holder portion with respect to the receiving portion, and wherein the opening is formed such that the needle portion can enter the housing and the needle holder portion can enter the receiving portion by means of a relative movement between the housing and the needle portion which relative movement is lateral with respect to the axis of the needle portion.

The present invention relates to an apparatus, a system and a method for achieving the safe removal of needles and in particular, though not necessarily, of hypodermic needles from injecting devices.

A range of drug injecting devices are currently available which comprise an outer re-useable housing into which is loaded a single or multi-dose drug containing cartridge. A hypodermic needle assembly is then attached to the cartridge through an end of the housing. An example of such a device is the Autopen™ injecting device available from Owen Mumford Ltd. of Woodstock, UK (see http://www.owenmumford.com). This device allows the dose to be delivered to be set using a dial mechanism. The force required to eject the drug from the cartridge is provided by a spring mechanism.

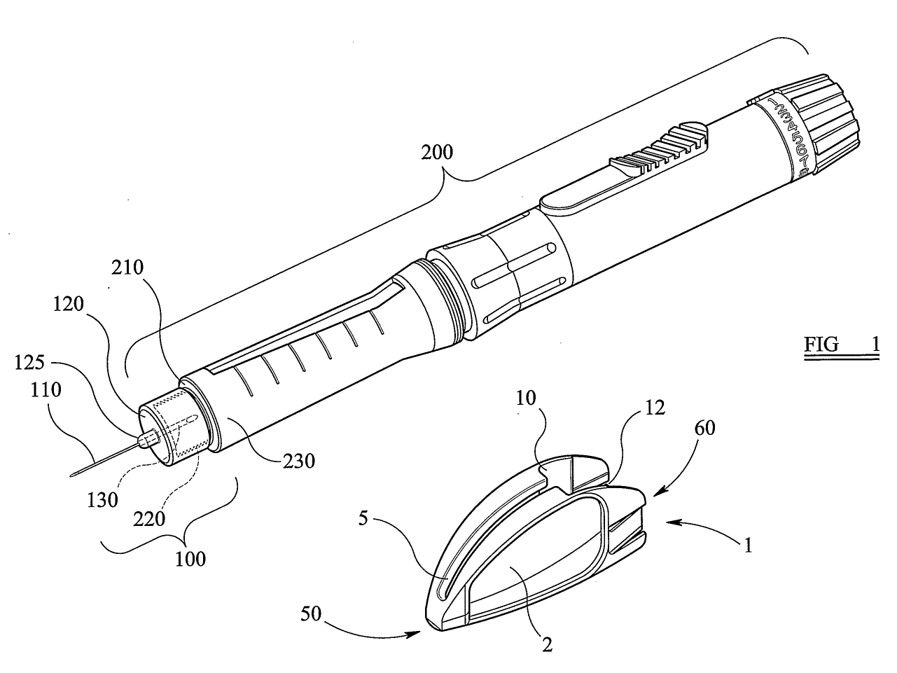

Considering in more detail the needle assemblies used with these injecting devices, a typical needle assembly is shown in FIG. 1 as 100 (top half of the figure). This needle assembly 100 comprises a stainless steel hypodermic needle 110 which is moulded into a generally cup-shaped plastics carrier 120, often referred to as a “hub”. The needle 110 extends axially through the hub 120 so that a shorter portion 130 (shown in dashed lines in FIG. 1) is contained within the hub 120 whilst a longer portion projects outwardly. A screw thread is formed around the inner surface of the hub. This thread engages a corresponding external thread on an end of the injecting device containing the cartridge (both threads are indicated in dashed lines as 220 in FIG. 1). The action of coupling the screw threads causes the end of the needle 130 within the cup to penetrate a membrane provided on the cartridge, thereby coupling the drug contents to the passage extending through the needle. FIG. 1 also shows a typical injecting device 200 to which the disposable needle arrangement 100 has been mounted. Needle assemblies of the type shown in FIG. 1 are available from Owen Mumford Ltd. of Woodstock, UK (http://www.owenmumford.com).

Used needles should be disposed of safely. The disposal should be simple and easy to encourage safe disposal. The disposable needle assemblies 100 are normally sold with a protective cap (not shown) which is pushed over the small protrusion 125 forming part of the hub 120 and being located at the distal end of the hub 120 (in this specification the axial ends of the various devices shown in the drawings will be referred to as “distal end” and “proximal end” whereby the distal end is shown at the left in FIG. 1 and the proximal end is shown at the right in FIG. 1). Further, the entire needle assembly (with protective cap in place) is usually packaged in a sterile container which is pushed over hub 120 and extends axially in distal direction so as to protect the needle and cap. The proximal end of the container is foil-sealed.

In use, a user removes the foil seal and threads the hub onto the injecting device. The user then removes the container and protective cap and then performs an injection. After the injection, in many instances users push the container back onto the hub 120 by means of a relative axial movement and then rotate the injecting device 200 with respect to hub 120 so as to loosen the threaded connection. The needle assembly 100 within the container can then be disposed of. However, the relative axial movement between the needle assembly 100 and the protective cap often results in injury if needle 110 and the container are not properly aligned.

The present invention has been made in the light of this problem. The present inventors have devised an apparatus which enables the needle arrangement 100 to be re-capped without using the axial movement used in the past.

Accordingly, the present invention provides apparatus for removing a needle assembly from an injecting device, the needle assembly comprising a needle holder portion and a needle portion secured to, or integral with, the needle holder portion, the needle assembly being attachable to the injecting device via the needle holder portion, the apparatus comprising
a housing having an opening through which the needle assembly can enter the interior of the housing, wherein within the housing there is provided a receiving portion which is arranged to receive the needle holder portion in such a way as to impede rotation of the needle holder portion with respect to the receiving portion, and wherein the opening is formed such that the needle portion can enter the housing and the needle holder portion can enter the receiving portion by means of a relative movement between the housing and the needle portion which relative movement is lateral with respect to the axis of the needle portion.

The present invention also provides a system comprising the above apparatus and a said needle assembly.

The invention further provides a method of sheathing a needle assembly attached to an injecting device, comprising:
providing the above system;
performing a relative movement between the housing and the needle portion which relative movement is lateral with respect to the axis of the needle portion so that the needle assembly enters the interior of the housing, and so that the needle holder portion enters the receiving portion by means of said relative movement.

Method aspects corresponding to apparatus aspects disclosed herein are also provided, and vice versa.

Some preferred embodiments of the invention will now be described by way of example only and with reference to the accompanying drawings, in which:

FIG. 1 shows a perspective view of an injecting device to which a needle assembly has been mounted, and an embodiment of the apparatus of the present invention.

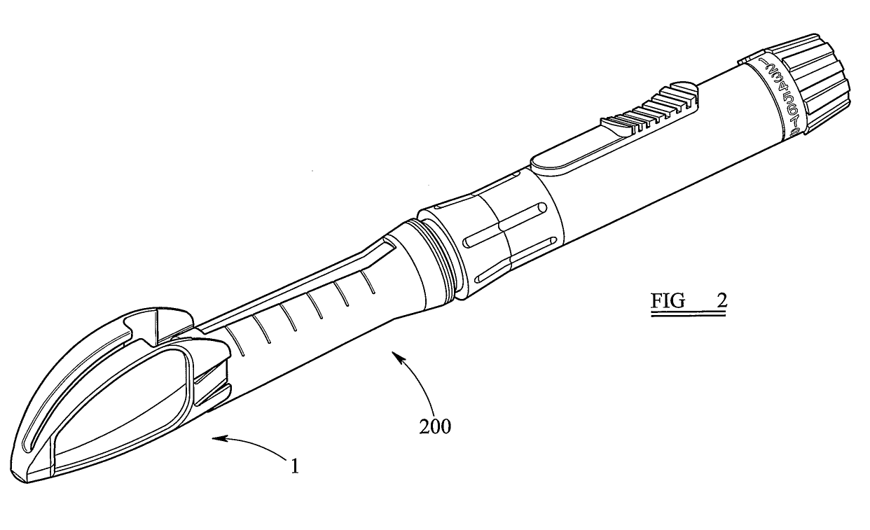

FIG. 2 shows the injecting device and needle assembly of FIG. 1 inserted into the apparatus of the present invention shown in FIG. 1.

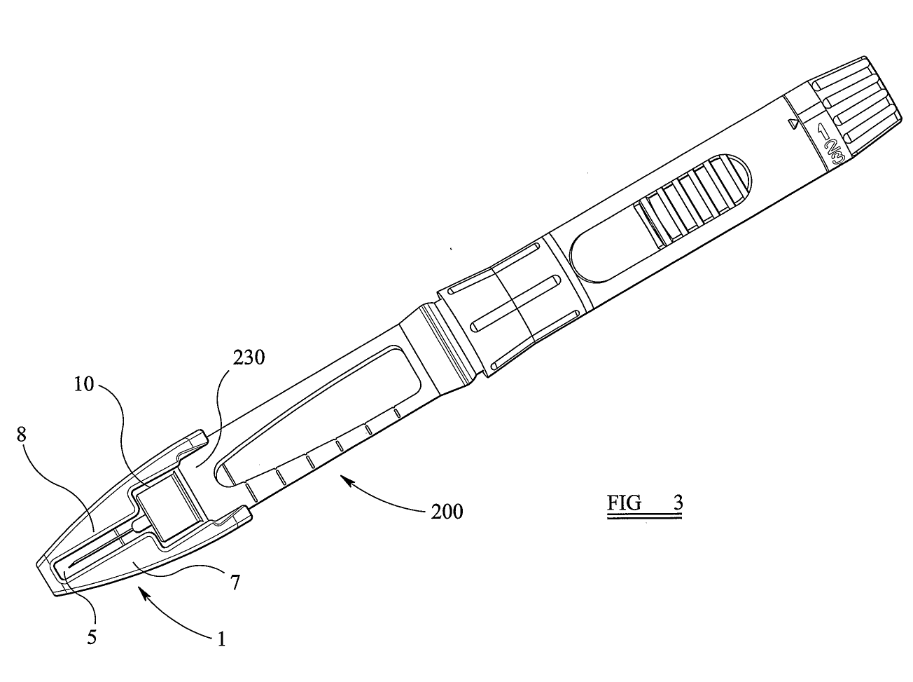

FIG. 3 shows a top view of the items shown in FIG. 2.

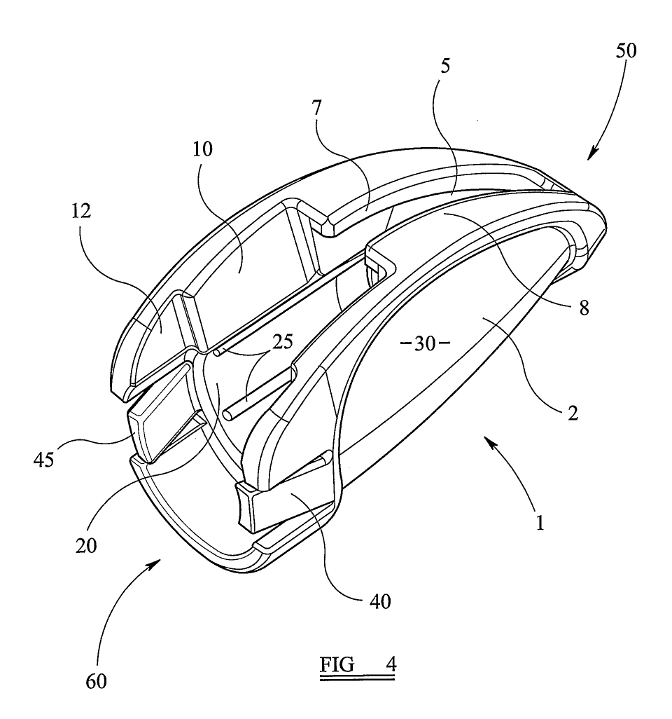

FIG. 4 shows a perspective, more detailed view of an embodiment of the apparatus of the present invention.

FIG. 5 shows a top view of the embodiment of FIG. 4.

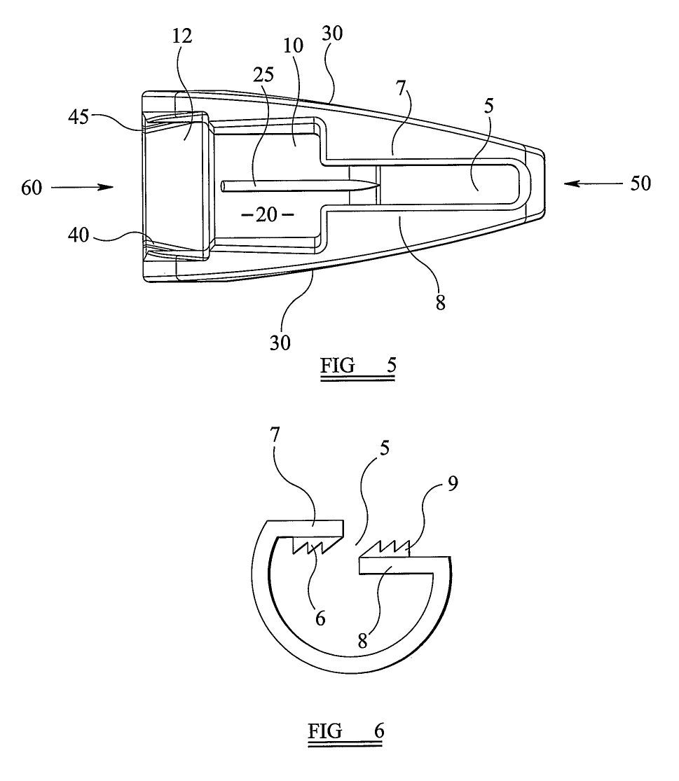

FIG. 6 shows a simplified cross-sectional view of a second embodiment of the present invention.

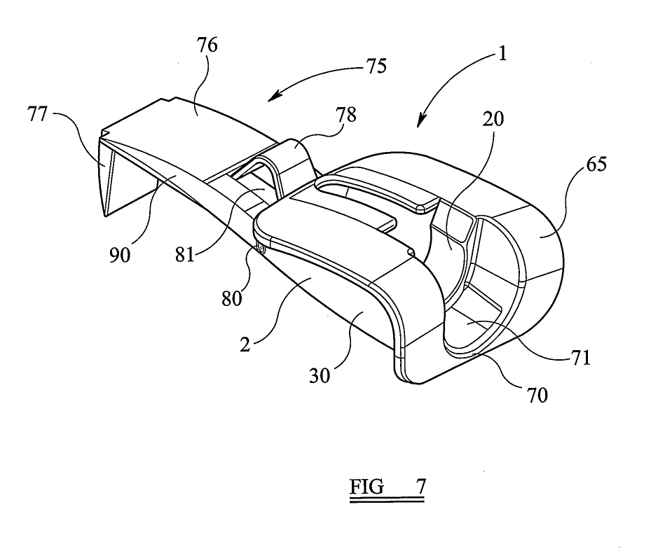

FIG. 7 shows a perspective view of a third embodiment of the apparatus of the present invention, in a first, open configuration.

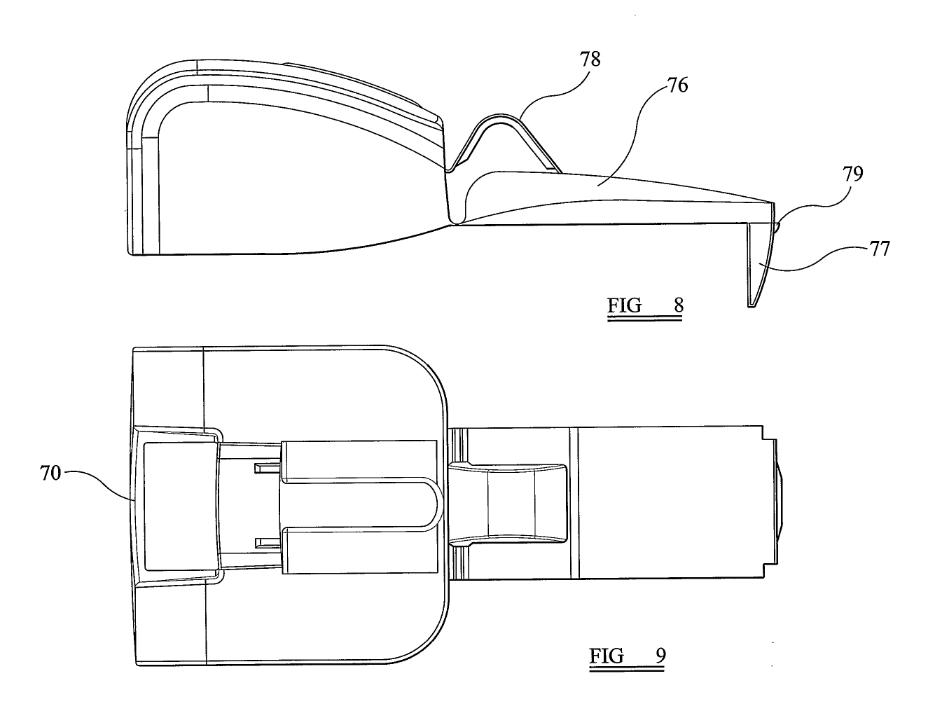

FIG. 8 shows a side view of the embodiment of FIG. 7.

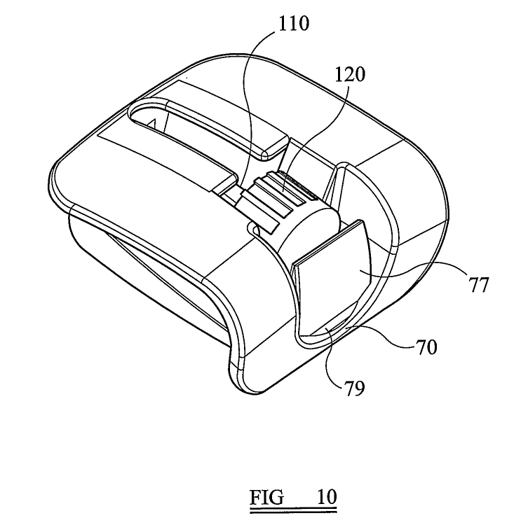

FIG. 9 shows a top view of the embodiment of FIG. 7.

FIG. 10 shows a perspective view of the embodiment of FIG. 7, in a second, closed configuration.

FIG. 1 shows a conventional injecting device 200 to which is mounted a needle assembly 100 comprising a cup-shaped hub 120 with a small protrusion 125 at the distal end of the hub 120 and carrying a needle 110. The hub 120 is formed with an internal thread 220 by means of which the hub 120 is threaded onto injecting device 200 which is formed with a corresponding external thread 220 at its distal end. “Behind” the threaded portion 220 of injection device 200 (i.e. adjacent to the threaded portion 220 in proximal direction) is a front portion 230 of the injecting device 200, which has a slightly larger diameter than the threaded portion 220. There is substantially no gap between the proximal end surface of the needle assembly 100 and the distal end surface 210 of front portion 230 of the injecting device 200, i.e. for practical purposes the proximal end surface of the needle assembly 100 lies flush against surface 210, so it is practically impossible for any tools to be applied between the proximal end surface of the needle assembly 100 and the distal end surface 210 of the enlarged front portion 230 of injecting device 200).

FIG. 1 also shows an embodiment of the apparatus 1 of the present invention. Apparatus 1 is generally oblong to suit the dimensions of a needle assembly with which it is intended to be used. Apparatus 1 comprises a housing 2, which is preferably moulded in one piece from a slightly elastic plastics material, such as polypropylene, polyethylene, acetal or ABS. The housing 2 is essentially closed at its distal axial end 50 and open at its proximal axial end 60. It is further formed with a lateral slot 5, 10, 12 which has a relatively narrow slot portion 5 towards the distal end of apparatus 1, a wider slot portion 10 generally in the middle of apparatus 1 and an even wider slot portion 12 towards the proximal end of apparatus 1. The slot portions 5, 10 and 12 and the axial opening at the proximal end of the apparatus 1 form a continuous opening. The distal slot portion 5 is slightly longer than the exposed portion 110 of the needle, the middle slot portion 10 is approximately of the same length in axial direction as the hub 120, and the proximal slot portion 12 is approximately of equal length as slot portion 10, although various other lengths can be used.

In the intended use of the present invention the injecting device 200 with mounted needle assembly 100 is approached to the apparatus 1 by means of a relative movement which is lateral with respect to the axis of needle 110. By means of this lateral movement, the needle assembly 100 and a portion of front portion 230 of injecting device 200 enters the housing 2 laterally until the injecting device 200 with mounted needle assembly 100 is secured to apparatus 1 as shown in FIG. 2. The relative movement is preferably such that the needle 110 passes through distal slot portion 5, hub 120 passes through middle slot portion 10 and a portion of front portion 230 of injecting device 200 passes through proximal slot portion 12 of housing 2. FIG. 3 shows a top view of the injecting device 200 with mounted needle assembly 100 secured to apparatus 1.

An industry standard for the external thread 220 of injecting device 200 has an overall diameter of about 9 mm. The external diameter and any surface features of hub 120 vary from manufacturer to manufacturer. Needle assemblies sold by the Applicant have a hub with an external diameter of about 11.5 mm with a plurality of longitudinally extending shallow grooves arranged around the circumference of the hub. One embodiment of the apparatus 1 of the present invention has a distal slot portion 5 which is about 3 to 4 mm wide, a middle slot portion 10 which is approximately 11 mm wide and a proximal slot portion 12 which is approximately 12 to 13 mm wide. These dimensions may ensure that the needle assembly 100 can be inserted into the apparatus 1 with relative ease, whilst ensuring that it is virtually impossible for a user's fingers to enter the distal slot portion 5.

Referring now to FIGS. 4 and 5, the internal structure of apparatus 1 is shown in more detail. A generally cylindrical receiving portion 20 is formed in the housing at the same axial position as middle slot portion 10. This receiving portion 20 is intended to receive, surround and grip hub 120 so as to impede rotation of hub 120 with respect to the receiving portion. During insertion of needle assembly 100 into the apparatus 1 by means of the lateral movement the hub passes through middle slot portion 10, and moves further into receiving portion 20 by means of the lateral movement. The width of middle slot portion 10 is preferably slightly smaller than the diameter of receiving portion 20 so as to improve retention of hub 120 within housing 1. As the dimensions of the outer diameter of hub 120 vary from one manufacturer to the other, the housing 2 is preferably slightly elastic. This enables the middle slot portion 10 to be slightly dilated during lateral entry of hub 120.

The elasticity of housing 2 also enables sides 30 to be squeezed together slightly. This may further improve the grip of hub 120 within receiving portion 20. This can be of particular significance if the hub 120 has a particularly small external diameter. The elasticity of housing 2 also enables the apparatus of the present invention to be used in conjunction with injecting devices 200 which have a relatively large external diameter of front portion 230 as proximal slot portion 12 can also be dilated elastically to a certain extent.

Receiving portion 20 has further at least one, but preferably several surface features which improve the grip of hub 120 within receiving portion 20. One such possible surface feature is shown in FIG. 4 as longitudinal ribs 25. These may engage with any surface features on hub 120, such as grooves, or ribs. A variety of other surface features 25 may be provided, including grooves or a matrix of dots.

It is to be noted that, upon proper insertion of needle assembly 100 the needle assembly 100 is entirely surrounded by housing 2 (although of course the opening 5, 10, 12, remains open, but no portion of needle assembly 100 would project out of a fully convex surface wrapped around apparatus 1 after insertion of needle assembly 100 into housing 2).

In proper use, once the hub 120 is located in receiving portion 20, the housing 2 can be held by one hand of the user and the injecting device 200 by the other hand of the user, and the apparatus 1 rotated with respect to injecting device 200 so as to loosen the threaded connection between the hub and the injecting device. Since the receiving portion 20 prevents (or at least impedes) rotation (about the axis of the needle assembly 100) of hub 120 with respect to housing 2, the needle assembly 100 remains (relatively) stationary with respect to housing 2. Once the threaded connection between hub 120 and injecting device 200 is fully undone the injecting device 200 can be moved in proximal axial direction so as to retract front portion 230 of injecting device 200 out of proximal slot portion 12 of apparatus 1. The needle assembly 100 is thus separated from injecting device 200 and remains within apparatus 1. Assuming that the dimensions of hub 120 and receiving portion 20 are chosen suitably, the needle assembly 100 will be held securely within apparatus 1 and apparatus 1 can be disposed of, together with needle assembly 100 held within its housing 2.

Preferably, a relatively sharp edge is provided at the transition between middle slot portion 10 and receiving portion 20. This further improves the grip of hub 120 within receiving portion 20 and may help to prevent accidental or deliberate removal of needle assembly 100 from within housing 2 (it will be appreciated that removal of needle assembly 100 from housing 2 could present a health risk, both in terms of injury through sharp needle 110 and infection from a used needle).

A further feature of the embodiment shown in FIG. 4 hinders removal of needle assembly 100 from apparatus 1: whilst at the proximal slot portion 12 the housing is formed generally cylindrical (the proximal slot portion 12 being provided in the lateral surface of the cylinder), two “legs” 40 and 45 are provided as resiliently deformable, radially inwardly projecting portions. The distal end of legs 40 and 45 is secured to receiving portion 20, while the proximal end of legs 40 and 45 projects inwardly. In the relaxed state of the apparatus 1 (i.e. without deformation caused by external forces or by insertion of injecting device 200) the distance between the proximal ends of legs 40 and 45 is smaller than the outer diameter of front portion 230 of a standard injecting device. On the other hand, the legs 40 and 45 are outwardly displaceable, (or pivotable) so that they are biased towards each other.

In normal use, insertion of injecting device 200 with needle assembly 100 into apparatus 1 by means of a lateral relative movement displaces legs 40 and 45 outwardly. The resulting bias of legs 40 and 45 does substantially not impede lateral entry of front portion 230 of injecting device 200 into proximal slot portion 12. Retraction of the injecting device 200 from apparatus 1 by unthreading the connection between hub 120 and injecting device 200 is also not substantially impeded. However, once the injecting device 200 is separated from hub 120 held within housing 2 legs 40 and 45 relax inwardly to their initial position, and since the distance between the proximal ends of legs 40 and 45 is slightly smaller than the outer diameter of front portion 230 of injecting device 200 any axial re-connecting of injecting device 200 to hub 120 is made substantially difficult.

It will be appreciated that only one, or more than two legs or other projections may be provided. In general terms, the diameter of the largest circle that can be placed between the proximal ends of the two or more legs 40, 45 should be smaller than the external diameter of the front portion 230 of injecting device 200.

In view of the different dimensions of hubs produced by various manufacturers, and further in view of the fact that the present invention may also find application with the removal of other needles, (such as the removal of needles from “traditional” syringes) it may be desirable to make apparatus 1 elastic to such an extent that it can be substantially deformed. The embodiment shown in FIG. 4 may not allow a large degree of deformation since upon lateral pressure onto sides 30 the opposing lips 7 and 8 forming distal slot portion 5 will abut so as to close distal slot portion 5, after which no further deformation can be achieved by pressure onto sides 30. To this end it is possible, according to a second embodiment, to provide the lips 7 and 8 at different radial positions. As a result, sides 30 can be squeezed further, with lips 7 and 8 overlapping. This is illustrated in the simplified cross section shown in FIG. 6. As a further refinement, and so as to aid in permanently gripping needle assembly 100 within housing 2 an engaging means may be provided so that lips 7 and 8 are retained permanently in the overlapping position. Such an engaging means is shown in FIG. 6 as two opposing ratchets 6, 9, one on each lip 7, 8.

Whilst it is preferred that the apparatus 1 of the present invention is a unitary moulded article (i.e. moulded in one piece), it is also possible to manufacture it from two or more different materials. For example, receiving portion 20 can be lined or otherwise provided with a grip enhancing material such as rubber, so as to increase the friction between hub 120 and receiving portion 20 (so as to aid in the prevention of rotation between hub 120 and receiving portion 20).

A third embodiment is illustrated in FIGS. 7 to 10. The general principle of this embodiment is essentially the same as that of the first embodiment, i.e. the apparatus 1 comprises a housing 2 with sides 30 and receiving portion 20 for receiving hub 120 of a needle assembly 100.

FIG. 7 shows the apparatus of the third embodiment in an open configuration. If it is moulded from plastics material it could for example leave the mould in that configuration.

Whilst in the first embodiment the bottom portion of the housing (i.e. opposite the slot 5, 10, 12) is closed, it is substantially open in the third embodiment. In particular, the third embodiment is formed with an opening 71 between the receiving portion 20 and a bridge portion 70 joining the sides 30 at the proximal end. However, an arm 75 is connected to the remainder of the housing at the distal end by means of e.g. a film hinge 80. The arm 75 comprises a relatively flat portion 76 which extends primarily in axial direction and, joined onto the distal end of portion 76 at approximately right angle thereto, a shorter portion 77. Portion 76 as shown in FIG. 7 will be referred to as “horizontal portion” and portion 77 will be referred to as “vertical portion”.

Arm 75 can be pivoted about hinge 80 by approximately 180° in counter-clockwise direction (as seen in FIG. 7) so that it substantially covers the open bottom of housing 2, as illustrated in FIG. 10. As shown in FIG. 10, when arm 75 is pivoted towards the closed configuration shown in FIG. 10, vertical portion 77 projects through opening 71 so as to block axial access to hub 120 from the proximal end.

Arm 75 is further formed with side portions 90 also extending at approximately 90° from horizontal portion 76, along the lateral edges of horizontal portion 76. This not only provides arm 75 with some degree of stability, but also complements the shape of sides 30 of housing 2 so that, in the closed configuration of FIG. 10, sides 30 and side portions 90 form a substantially smooth surface which closes the apparatus laterally.

Arm 75 is further provided with an opening 81 at the centre of its proximal end (as seen in FIG. 7). A tongue 78 extends between the proximal and distal end of opening 81. At its distal end, tongue 78 is joined onto horizontal portion 76, whereas its proximal end is joined onto the distal end of housing 2 (approximately in line with hinge 80). The connection between tongue 78 and horizontal portion 76 on the one hand and housing 2 on the other hand is also achieved by means of a film hinge. As best seen in FIG. 8, tongue 78 has a generally “V” shape section. That is, the crest of this “V” shape projects upwards (as seen in FIG. 7) when the apparatus is in the open configuration. Tongue 78 is “bistable”, i.e. in the absence of external forces the apparatus 1 would remain in the open configuration shown in FIGS. 7 to 9, but, once brought into the closed configuration shown in FIG. 10, the apparatus 1 would also remain in that configuration, in the absence of external forces.

Once the apparatus has been brought into the closed configuration the tongue 78 approximately covers the entire opening 81. Preferably, any gap between the sides of tongue 78 and those portions of horizontal portion 76 which are located adjacent the opening 81 is narrower than the diameter of a typical needle 110.

Finally, arm 75 is formed with a locking projection 79 (best seen in FIG. 8), where the horizontal and vertical portions 76, 77 join. The generally wedge shaped locking projection 79 projects slightly from the vertical portion 77 in distal direction (as seen in FIG. 8). As the apparatus 1 is transferred from the open configuration into the closed configuration the vertical portion 77 moves past bridge 70 with only a minimal gap therebetween. The pivoting movement of arm 75 is temporarily stopped when locking projection 79 “strikes” bridge 70 from underneath. The biasing force exerted by tongue 78 is not strong enough to force locking projection 79 past bridge 70, i.e. once most of the pivoting movement has been performed, the arm 75 would remain in an intermediate configuration (not shown in the drawings) where locking projection 79 buts against bridge 70 from underneath. Only once sufficient force is exerted onto the apparatus, e.g. by holding the apparatus 1 between a user's thumb and index and middle fingers (the thumb pressing upwards onto the horizontal portion 76 and the index and middle fingers pressing downwards onto the top of housing 2, adjacent the slot 5, 10, 12). When sufficient force is exerted the bridge 70 slides past locking projection 79, using the wedge shaped locking projection 79 as a ramp. Once this sliding movement has been completed the apparatus is in the closed configuration shown in FIG. 10. The sliding movement is completed by locking projection 79 “clicking” into place once it has passed bridge 70, and bridge 70 cannot then easily slide back past locking projection 79 since locking projection 79 is formed with a ramp only on one side.

As a further difference to the first embodiment, the third embodiment is formed with a finger protection 65 which, in the embodiment shown in the drawings, comprises a flange adjacent to the slot 5, 10, 12 and the proximal opening. When apparatus 1 is held between a user's thumb and index finger (the two sides 30 of housing 2 between thumb and index finger) the flange 65 covers a substantial portion of the user's fingers so as to protect the user against injury during the insertion process.

The insertion process of the third embodiment can be carried out as follows. The apparatus 1 may be supplied in the intermediate configuration (not shown), i.e. the configuration where the locking projection 79 buts against bridge 70 from underneath. Vertical portion 77 partially obstructs the axial opening of housing 2 at its proximal end. The user would then hold housing 2 between thumb and index finger of one hand whilst holding an injecting device 200 with mounted needle assembly 100 in the other hand. The needle assembly 100 and the front portion of the injecting device 200 is then inserted through slot 5, 10, 12 into housing 2 as has been described in connection with the first embodiment. As hub 120 enters the receiving portion 20 the front portion 230 of injecting device 200 pushes down onto vertical portion 77 so as to pivot arm 75 away from housing 2. This can be done with relative ease since only the relatively weak biasing force of tongue 78 needs to be overcome. However, during insertion of needle assembly 100 and front portion 230 of injecting device 200 arm 75 remains biased towards the closed configuration, i.e. it is not “flipped” into the open configuration.

Once hub 120 has been received in receiving portion 20 the injecting device 200 is separated from needle assembly 100 by unthreading the threaded connection and axially withdrawing the injecting device 200 in proximal direction relative to needle assembly 100. If necessary, the grip between receiving portion 20 and hub 120 can be increased by the application of lateral pressure onto sides 30. As injecting device 200 is withdrawn axially it no longer pushes down onto vertical portion 77 so that the apparatus returns to the intermediate configuration where locking projection 79 buts against bridge 70 from underneath. The user can then bring apparatus 1 into the closed configuration by squeezing the apparatus 1 between thumb, index and middle finger, as described above. Once locking projection 79 has moved past bridge 70 axial access to hub 120 is substantially blocked. Hence it is virtually impossible to reconnect needle assembly 100 and injecting device 200. Needle assembly 100 is then held securely within closed housing 2 and can safely be disposed of, together with apparatus 1.

It will be appreciated that, whilst the first to third embodiments have been described separately, various features described in connection with one particular embodiment may be included in any of the other embodiments.

Although the invention has been described in terms of preferred embodiments as set forth above, it should be understood that these embodiments are illustrative only and that the claims are not limited to those embodiments. Those skilled in the art will be able to make modifications and alternatives in view of the disclosure which are contemplated as falling within the scope of the appended claims. Each feature disclosed or illustrated in the present specification may be incorporated in the invention, whether alone or in any appropriate combination with any other feature disclosed or illustrated herein.

1. Apparatus for removing a needle assembly from an injecting device, the needle assembly comprising a needle holder portion and a needle portion secured to, or integral with, the needle holder portion, the needle assembly being attachable to the injecting device via the needle holder portion, the apparatus comprising
a housing having an opening through which the needle assembly can enter the interior of the housing, wherein within the housing there is provided a receiving portion which is arranged to receive the needle holder portion in such a way as to impede rotation of the needle holder portion with respect to the receiving portion, and wherein the opening is formed such that the needle portion can enter the housing and the needle holder portion can enter the receiving portion by means of a relative movement between the housing and the needle portion which relative movement is lateral with respect to the axis of the needle portion.

2. Apparatus according to claim 1, wherein the opening comprises a slot at one side of the housing.

3. Apparatus according to claim 2, wherein the slot is of non-uniform width.

4. Apparatus according to claim 3, wherein the slot comprises a first slot portion through which the needle portion is arranged to pass, and a second slot portion through which the needle holder portion is arranged to pass, wherein the second slot portion is wider, preferably substantially wider than the first slot portion.

5. Apparatus according to claim 4, wherein the second slot portion is at least twice as wide as the first slot portion.

6. Apparatus according to claim 5, wherein the second slot portion is at least 8 mm wide, preferably at least 9 mm wide.

7. Apparatus according to claim 4, wherein the first slot portion is less than 5 mm wide, preferably less than 4 mm wide.

8. Apparatus according to claim 1, wherein the apparatus is resiliently deformable.

9. Apparatus according to claim 8, wherein, the opening comprises a slot at one side of the housing, and wherein, in the relaxed state of the apparatus, the slot is open sufficiently wide so as to permit the needle portion and the needle holder portion to pass through the slot.

10. Apparatus according to claim 8, wherein the apparatus is arranged to deform resiliently as the needle holder portion enters the receiving portion.

11. Apparatus according to claim 8, wherein rotation of the needle holder portion with respect to the receiving portion can be impeded or further impeded by the application of lateral pressure onto the apparatus.

12. Apparatus according to claim 1, wherein the housing is formed so as to be able to surround the entire needle holder portion.

13. Apparatus according to claim 1, wherein the housing is formed so as to be able to surround the entire needle assembly.

14. Apparatus according to claim 1, wherein the needle holder portion is formed with an internal thread for connection to an external thread formed on the injecting device.

15. Apparatus according to claim 14, wherein the thread of the injecting device is a standard thread for insulin injecting devices.

16. Apparatus according to claim 14, wherein the thread of the injecting device is a thread of the type suitable for use with insulin injecting devices complying with ISO 11608.

17. Apparatus according to claim 14, wherein the thread of the injecting device has an external diameter of 9.5 mm and has a pitch of 32 threads per inch (32 threads per 2.54 cm).

18. Apparatus according to claim 1, wherein the external diameter of the needle holder portion is between 10 and 14 mm, preferably between 10 and 13 mm, more preferably between 10 and 12 mm and most preferably between 11 and 12 mm.

19. Apparatus according to claim 1, wherein the receiving portion is formed with at least one longitudinal rib for inhibiting said rotation.

20. Apparatus according to claim 1, wherein the receiving portion is formed with a friction-increasing material for inhibiting said rotation.

21. Apparatus according to claim 20, wherein at least one portion of the apparatus other than said receiving portion is formed with a material which has a lower coefficient of friction than said friction-increasing material.

22. Apparatus according to claim 1, wherein the apparatus is a unitary moulded article.

23. Apparatus according to claim 1, comprising first impeding means for impeding removal of the needle assembly from the apparatus.

24. Apparatus according to claim 23, wherein the first impeding means comprises a neck portion between the opening and the receiving portion which has a smaller passage width than the receiving portion.

25. Apparatus according to claim 1, further comprising second impeding means for impeding re-attachment of the injecting device to the needle holder portion once the needle assembly has been removed from the injecting device.

26. Apparatus according to claim 25, wherein the second impeding means comprises at least one radially inwardly projecting portion located towards one axial end of the apparatus.

27. Apparatus according to claim 26, wherein the inwardly projecting portion is displaceable outwardly so that it is biased inwardly.

28. Apparatus according to claim 27, wherein the second impeding means comprises at least two such radially inwardly projecting portions generally opposing each other.

29. Apparatus according to claim 28, wherein the largest circle which can be placed between said inwardly projecting portions in their relaxed state has a diameter which is smaller than the diameter of that portion of the injecting device which is immediately adjacent to that portion of the injecting device to which the needle holder portion is arranged to be attached.

30. Apparatus according to claim 25, wherein the second impeding means comprises means for substantially blocking access to the proximal end of the needle holder assembly in axial direction when the needle holder portion has been received in the receiving portion.

31. Apparatus according to claim 30, wherein the blocking means comprising a member which is connected to the remainder of the apparatus by means of a hinge so that it is movable between a first position in which it does not block said access and a second position in which it does block said access.

32. Apparatus according to claim 31, wherein the blocking means comprises locking means for locking the member in the second position.

33. Apparatus according to claim 2, wherein at least a portion of the slot is formed by two generally opposing lips located at different radial positions of the apparatus.

34. Apparatus according to claim 33, wherein the apparatus can be squeezed such that the two lips overlap.

35. Apparatus according to claim 34, further comprising engaging means for engaging the lips with each other when they have been caused to overlap.

36. Apparatus according to claim 1, further comprising a shield for protecting the fingers of a user when holding the apparatus.

37. Apparatus according to claim 36, wherein the shield comprises a flange adjacent the opening.

38. A system comprising the apparatus of claim 1 and a said needle assembly.

39. A system according to claim 38, further comprising a said injecting device to which said needle assembly is attached.

40. A method of sheathing a needle assembly attached to an injecting device, comprising:
providing a system according to claim 39;
performing a relative movement between the housing and the needle portion which relative movement is lateral with respect to the axis of the needle portion so that the needle assembly enters the interior of the housing, and so that the needle holder portion enters the receiving portion by means of said relative movement.

41. A method of removing a needle assembly from an injecting device, comprising:
performing the method according to claim 40;
rotating the apparatus with respect to the injecting device; and
removing the apparatus axially from the injecting device so as to axially remove the needle assembly from the injecting device.

42. A method according to claim 40, wherein, when the needle holder portion is attached to the injecting device, there is substantially no gap between the injecting device and that surface of the needle holder portion which is furthest away from the tip of the needle portion.
